(19) United States
(12) Patent Application Publication
(10) Pub. No.: US 2012/0103831 A1
(43) Pub. Date: May 3, 2012

(54) METHOD FOR MEMBRANE PERMEABILIZATION OF BIOLOGICAL CELLS BY USING A PULSED ELECTRIC FIELD

(75) Inventors: Luc Schrive, Pont Saint Esprit (FR); Guy Lumia, Pont Saint Esprit (FR); Florian Pujol, Montpellier (FR)

(73) Assignee: Commissariat a renergie atomique et auxqenergies aux energies altenatives, Paris (FR)

(21) Appl. No.: 13/262,222

(22) PCT Filed: Apr. 6, 2010
(86) PCT No.: PCT/EP2010/054511

§ 371 Date: Dec. 16, 2011

(30) Foreign Application Priority Data
Apr. 7, 2009 (FR) 09 52262

(51) Int. Cl. A23L 2/50; C02F 1/48; C12N 13/00; A23L 3/32

(52) U.S. Cl. 205/701

(57) ABSTRACT

A method for membrane permeabilization of biological cells contained in a product is disclosed. According to one aspect, the method is applied in a treatment device including at least one treatment chamber emitting a pulsed electric field. According to another aspect, the method includes supplying the treatment device with a product including biological cells at a predetermined supply flow rate from a supply unit, introducing the product including the biological cells into the treatment chamber at an introduction flow rate, treating the product introduced into the chamber with a pulsed electric field, and reintroducing the treated product, at a predetermined feedback flow rate at a point that is downstream from the supply unit. According to some aspects, the introduction flow rate includes the feedback flow rate and the supply flow rate.

### TECHNICAL FIELD

The present invention relates to a method for membrane permeabilization of biological cells by using a pulsed electric field.

This method most particularly finds its application in the field of pasteurization, i.e. the field for treating food products (such as milk, creams, beer, fruit juices), consisting of destroying microorganisms, notably pathogens initially present in these products.

The field of the invention is therefore that of membrane permeabilization of biological cells and, in particular, of destruction of biological cells by pasteurization.

### STATE OF THE PRIOR ART

Pasteurization techniques have been the subject of many studies in the prior art, notably with regard to the development of long-life products, which require very low presence of microorganisms for being able to be consumed in the long term.

Conventionally, pasteurization consists of heating the foodstuffs to a defined temperature for a defined period so as to exceed the heat-resistance threshold of pathogenic bacteria being at the origin of deterioration of the foodstuffs and then of rapidly cooling said heated foodstuffs at temperatures (from 3 to 4° C.), so as to prevent multiplication of the bacteria which have not been destroyed.

This conventional principle has been the subject of many alternatives belonging to the category of treatments, so-called “heat treatments”.

Heat treatments for pasteurization may consist in using as a heat vector the following means:

electromagnetic radiations, such as infrared radiations, microwave radiations;

heat originating from the Joule effect phenomenon generated in a tube in which flows the product to be pasteurized;

the ohmic heat resulting from an electric current passing through the product to be pasteurized.

The pasteurization temperatures attained via a thermal route conventionally range from 70° C. to 85° C. However, after treatment in these ranges of temperatures, there may subsist certain pathogenic forms such as spores incompatible for products intended for food.

In order to destroy these pathogenic forms, one of the solutions may consist of heating the foodstuffs to higher temperatures than the aforementioned range (for example at temperatures above 90° C.). However the use of higher temperatures is inevitably accompanied by denaturation of the treated product, such as denaturation of the proteins present in the product, which often does not occur without a loss of the gustatory properties of the product.

In order to find a remedy to these drawbacks, it was proposed to resort to so-called “low temperature” methods, so as to preserve the original taste of the product. These methods consist of resorting to means for removing pathogenic bacteria other than the use of heating, allowing treatment of foodstuffs at temperatures not exceeding 60° C. These means may consist in ionizing radiations, the use of high pressures, pulsed light, the use of a gas such as carbon dioxide.

Considering what has already been proposed as regards a membrane permeabilization method, and in particular a pasteurization method, the authors propose to develop a novel method for membrane permeabilization which notably allows, when it is applied with view to pasteurizing a food product, an improvement in the mortality rate of the undesirable cells.

### DISCUSSION OF THE INVENTION

Thus, the invention according to a first object relates to a method for membrane permeabilization of biological cells contained in a product, said method being applied in a treatment device comprising at least one treatment chamber emitting a pulsed electric field, said method comprising the following steps:

a step for supplying the treatment device with a product comprising said biological cells at a predetermined supply rate from a supply unit comprising said product;

a step for introducing the product comprising said biological cells in said treatment chamber at an introduction rate corresponding to the aforementioned supply rate to which is added the drawing-off rate mentioned in the drawing-off step below;

a step for treating said product introduced into said chamber with a pulsed electric field;

a drawing-off step, at a predetermined drawing-off rate, for the product at the outlet of said chamber so as to transport it again upstream from said chamber and downstream from said supply unit.

It is understood that the method of the invention is an in vitro method for membrane permeabilization of biological cells, i.e. which is applied with a product located outside an animal organism.

The inventors surprisingly noticed that the method of the invention gives the possibility of obtaining a significant increase in the membrane permeabilization of cells and this with at least one treatment chamber (the increase being quantified in terms of mortality rate of the cells) as compared with a method comprising one or more passages of the product in the aforementioned chamber without using a drawing-off step as mentioned above and this for a same specific energy used.

According to the invention, the cells treated within the scope of the method may be prokaryotic, eukaryotic cells, these cells may be live or dead, entire or partial, and cells of animal or vegetable origin.

In particular, these cells may stem from unicellular or pluricellular organisms, such as bacteria, (in particular, eubacteria), fungi (in particular, molds, yeasts (like Saccharomyces cerevisiae), molds, algae (in particular micro-algae), viruses and prions.

These may also be intracellular organites or compounds internal to the cells such as mitochondria, viruses, proteins, prions.

The cells may be in a vegetative phase or dormancy phase (as this may be the case for spores, such as bacterial spores).

The cells to be treated are comprised in a product, which may be a foodstuff liquid, such as fruit juice, vegetable juice, milk, water.

Further, the product comprising said cells may contain tissues, macromolecules, such as biopolymers, organic or inorganic molecules.

The pulsed electric field to which the product comprising the cells is subject, has the effect of permeabilizing said cells.

Without the intention of being bound by theory, exposure of a cell to an outer electric field induces an electric potential difference on either side of the constitutive membrane of the cell. When this field is very intense (notably when it is above 10,000 V/cm), it may induce a transmembrane potential of a higher value than the natural potential of the cell. When the transmembrane potential attains a critical value, electrostatic phenomena between the charged molecules on either side of the membrane cause the formation of pores in the cell membrane thereby increasing its permeability. When the formation of pores in the cell membrane is irreversible, this may cause migration outwards of the cell contents and thus the death of the cell.

According to the invention, the pulsed electric field, depending on the desired permeabilization level will have characteristics required for obtaining this permeabilization in terms of the voltage value, of the number of pulses, of the shape of the signal, of the specific energy delivered by said field.

The pulsed electric field is conventionally materialized in the form of electric pulses conventionally resulting from electric discharges with a period which may range from 50 nanoseconds to 1 millisecond, for example 1 μs and delivering a voltage producing a peak value of the pulsed electric field ranging from 5 kV/cm to 200 kV/cm. The voltage may range from 100 V to 100,000 V.

By using electric pulses it is possible to minimize the phenomenon of heating the product by the Joule effect and thus the temperature of the latter (conventionally less than 50° C.). With this, it is possible to avoid denaturation of components present in the product, which denaturation is conventionally encountered during the application of a method involving high temperatures.

The electric pulses are delivered within a treatment chamber which may have the form of an enclosure comprising electrodes which allow the current to pass as electric pulses. These electrodes may be planar, circular, co-axial, co-linear, rotary or have any other suitable geometry.

As announced earlier, the method comprises a step for supplying the treatment device with a product to be treated comprising said cells at a pre-determined supply rate from a supply unit comprising said product.

This step conventionally consists of having the product to be treated pass in transit from a supply unit, such as a tank, where it is contained towards a supply conduit which is connected to the treatment chamber.

From a practical point of view, the product to be treated feeds the treatment device from a supply unit via a supply conduit optionally provided with a supply pump at a predetermined supply rate, such as a rate ranging from 0.01 L/h to 10,000 L/h, for example a flow rate of 20 L/h.

Next, the method of the invention comprises a step for introducing the product comprising said biological cells into said treatment chamber at an introduction rate corresponding to the aforementioned supply rate to which is added the drawing-off rate mentioned in the drawing-off step below.

Once the product is introduced into the treatment chamber, it is subject to a pulsed electric field which is materialized by electric pulses, which will cause permeabilization of the cells contained in the liquid.

The electric pulses are advantageously pulses, which may range from 100 to 100,000 V, for example from 10,000 to 50,000 V and delivering sufficient energy in order to obtain the desired permeabilization according to the sought biological effect. The energy delivered by each pulse is conventionally comprised between 0.005 J and 500 J.

Thus, when it is desired to obtain permeabilization of the cells causing the death of the latter, (notably with view to pasteurization or sterilization of the product), the treatment parameters and, notably the total number of pulses delivered to each volume element (corresponding to the volume of the treatment chamber) will be selected so as to deliver to the product a specific energy greater than 30 MJ/m³, preferably greater than 100 MJ/m³.

To obtain the death of the cell, depending on the delivered energy at each pulse and depending on the volume of the treatment chamber, the total number of pulses delivered to each volume element (corresponding to the volume of the treatment chamber) will be adjusted in order to attain intended values of specific energy. This total number of pulses may be comprised between 10 and 1,000.

When it is desired to obtain permeabilization of the cells so as to accelerate exchanges between the cell and the surrounding medium, the electric pulses emitted by the treatment chamber may be selected so as to deliver to the product over the whole duration of the process, a specific energy of less than 20 MJ/m³, for example from 1 to 20 MJ/m³. The total number of pulses may be comprised between 2 and 100.

It is a matter of fact that one skilled in the art depending on the desired permeabilization rate will select the characteristics of the suitable electric pulses in terms of the number of pulses, of the voltage, of the delivered specific energy.

The number of pulses to be applied to each volume element may de distributed in several series.

As an example, when it is considered that 120 pulses are necessary for each volume element for treating the product), the total number of pulses to be applied, may be distributed, for example in 3 series, 6 series or 10 series, each series allowing the application of 40, 20, or 12 pulses respectively.

Inside the treatment chamber, the product, notably when it appears as a liquid, may be subject to turbulent hydraulic conditions. This hydraulic mode is particularly advantageous. Indeed, permeabilization of the cells during an electric discharge is preferentially carried out on the faces of the cell membrane located facing the electrodes. When a cell is in disordered motion (which is the case when the product is subject to turbulent conditions) relatively to the electrodes and is subject to a series of pulses, it will experience disseminated impacts, which will contribute to increasing the efficiency of the treatment in terms of permeabilization. Conversely, an immobile cell relatively to the electrodes will be subject to a concentration of the impacts, which will reduce the efficiency of the treatment.

Once the treatment step is achieved, according to the method of the invention, a drawing-off step is provided, downstream from the treatment chamber, for the treated product in the treatment chamber at a predetermined drawing-off rate, so as to again transport this drawn-off product upstream from the treatment chamber and downstream from said supply unit, whereby the drawn-off product is again introduced into the treatment chamber and again subject to the pulsed electric field.

According to a particular embodiment of the invention, the drawing-off step is achieved by means of at least one circulation loop connecting the downstream portion of the treatment chamber to the upstream portion of the latter. At the outlet of the treatment chamber, the treated product is drawn off via the inlet of a circulation loop located downstream from the chamber and injected upstream from the chamber via the outlet of the circulation loop, this circulation loop appearing as a circulation conduit.

The drawing-off rate may be greater than or equal to the aforementioned supply rate.

Advantageously, the drawing-off rate is greater than the supply rate, for example from 2 to 100 times higher, preferably 2 to 20 times higher. This allows each product fraction to pass at least twice through the treatment chamber.

The application of a drawing-off step according to the invention has the following advantages.

With this, it is possible to lessen the hydraulic short circuit phenomenon, i.e. the probability that one cell taken on the whole of the treated cell population passes more rapidly through the treatment chamber than the remainder of this population.

For a given specific energy, by applying the drawing-off step according to the invention, better results are obtained in terms of cell permeabilization than for a method having a first treatment of the liquid by causing it to pass into a treatment chamber followed by a second treatment in the same treatment chamber after having been stored in a tank between the first treatment and the second treatment.

Advantageously, the method does not comprise any step for stagnant storage of the treated product before the drawing-off step, i.e. before re-introducing the latter into the treatment chamber via the drawing-off step.

Another advantage comes from an unexpected effect related to the effect that the number of pulses delivered to each volume element is delivered in several times because of the existence of the recycling loop. It was observed surprisingly that for given hydraulic conditions in the treatment chamber and for a given total number of pulses, it was more advantageous to distribute this number in several times rather than subject the product to be treated to the total number of pulses in a single go, without using any recycling loop.

According to another embodiment of the invention, the drawing-off step may be carried out via several circulation loops, the inlets of which are located downstream from the treatment chamber and the outlets of which are located upstream from the treatment chamber.

The fact of establishing several circulation loops makes it possible to reduce the probability of cells passing into a hydraulic short-circuit situation, as defined above.

The method of the invention also advantageously comprises a step for extracting the treated product from the treatment device at an extraction rate advantageously corresponding to the aforementioned supply rate.

By extraction step is meant a step consisting in an extraction at the outlet of the device downstream from the chamber for treating the product, so as to avoid accumulation of product in the device. The extracted and treated product may be recovered in a collecting tank.

As mentioned above, the treatment device comprises at least one treatment chamber, which means that it may contain several of them.

In this case, the treatment chambers may be placed side by side, the cycle of steps, i.e. the introduction step, the treatment step and the drawing-off step, respectively, as explained above, taking place in each of said treatment chambers.

The method of the invention may be applied with a treatment device respectively comprising:

a unit for supplying the product to be treated, appearing for example as a tank, in which the product to be treated is contained;

at least one treatment chamber emitting a pulsed electric field connected to the supply unit via a supply conduit;

an outlet conduit located downstream from the treatment chamber;

at least one circulation loop, the inlet of which is located downstream from the treatment chamber and is connected to the outlet conduit and the outlet is located upstream from the treatment chamber and is connected to the supply conduit.

Pumps may be provided on the supply conduit and on the circulation loop.

The method of the invention when the drawing-off step is carried out via a single circulation loop, may be carried out from a treatment device 1, as illustrated in FIG. 1, comprising respectively:

a supply unit 2 comprising the product to be treated;

a supply conduit 3 connecting the supply unit to a treatment chamber 5, on the path of which may be interposed pumps, such as a main pump 7 and a secondary pump 9;

a treatment chamber 5 within which a pulsed electric field is delivered;

an outlet conduit 10 connecting the treatment chamber 5 to a tank 11 for receiving the treated liquid;

a circulation loop 13 materialized as a conduit, the inlet 15 of which is located downstream from the treatment chamber on the outlet conduit 10 and the outlet 17 is located upstream from the treatment chamber 5 on the supply conduit 3, this loop allowing transport of at least one portion of the treated product downstream from the treatment chamber so that this portion is again subject to a pulsed electric field.

The method of the invention, when the drawing-off step is performed via several circulation loops, may be carried out from a treatment device 1, as illustrated in FIG. 2, respectively comprising:

a supply unit 2, comprising the product to be treated;

a supply conduit 3 connecting the supply unit 3 to a treatment chamber 5, on the path of which may be interposed pumps, such as a main pump 7 and a secondary pump 9;

a treatment chamber 5 within which a pulsed electric field is delivered;

an outlet conduit 10 connecting the treatment chamber 5 to a tank 11 for receiving the treated liquid;

three circulation loops 15, 17 and 19 respectively materialized as conduits, the inlets 21, 23 and 25 of which are located downstream from the treatment chamber 5 on the outlet conduit 10 and the outlets 27, 29 and 31 are located upstream from the treatment chamber 5 on the supply conduit 3, these loops allowing transport of at least one portion of the treated product downstream from the treatment chamber so that this portion is again subject to a pulsed electric field, optionally via pump(s) which may be common to the whole of the circulation loops or independent for each of the loops.

In the circulation loop(s), may be inserted one or more apparatuses such as heat exchangers, material exchangers (for example phase separators, momentum exchangers (i.e., devices allowing modification and improvement of the circulation of the fluid, such as a pump, a mobile stirrer, a static stirrer).

The product to be treated according to the method of the invention may appear as a liquid comprising biological cells to be permeabilized, in the form of muds or pluricellular organisms, such as fruit.

When this is a liquid, this may notably be water, liquid effluents, liquid muds from sewage works, fruit juice, milk, liquid eggs, sauces, soups, stewed fruit and purees.

This may notably be a liquid comprising organites or molecules stemming from biological cells such as mitochondria, DNA or RNA.

When these are muds, these may notably be muds from sewage works.

The method of the invention may be intended for different uses, such as:

pasteurization or sterilization of liquids such as water, liquid effluents, fruit juices, milk, liquid eggs, sauces, soups, purees, stewed fruit and purees;

treatment of muds from sewage works, in order to remove certain pluricellular organisms and microorganisms, before spreading of these muds or dehydration before drying;

treatment of biological cells in the field of genetic engineering, with view to making them permeable to exogenous molecules (such as DNA, RNA, proteins, viruses);

breaking up cells of pluricellular organisms, such as fruit, algae, with view to facilitating their subsequent pressing in order to obtain a fruit juice or a fat extract.

The method of the invention is most particularly adapted to pasteurization or sterilization of a liquid.

Thus, the invention also relates to a method for pasteurization or sterilization of a product comprising the application of the method as defined below, the parameters of the pulsed electric field being set so as to obtain membrane permeabilization of the biological cells present in the liquid leading to the death of the latter.

This may notably be pasteurization or sterilization of a fruit juice, comprising, as biological cells, yeasts such as Saccharomyces cerevisiae.

The invention will now be described with reference to the following examples given as an illustration and not as a limitation.

### SHORT DESCRIPTION OF THE DRAWINGS

FIG. 1 illustrates an example of a treatment device including a circulation loop allowing the application of the method of the invention.

FIG. 2 illustrates an example of a treatment device including three circulation loops allowing application of the method of the invention.

FIG. 3 is a graph illustrating Log (N/N₀) versus the delivered specific energy (in MJ/m³) for the comparative Example (curve a) and the Example of the invention (curve b).

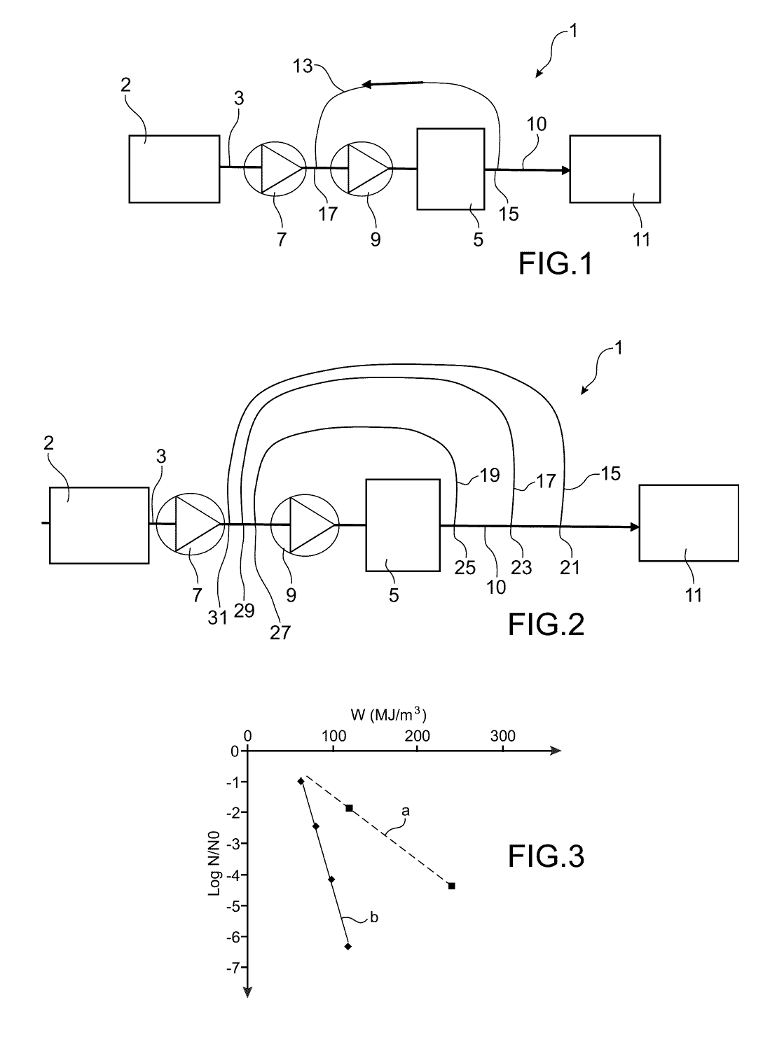

### DETAILED DISCUSSION OF PARTICULAR EMBODIMENTS

The following examples illustrate the application of a method not falling within the scope of the invention on the one hand (a so-called comparative Example) and of a method according to the invention (a so-called Example of the invention).

To do this, orange juice contaminated by Saccharomyces cerevisiae yeasts is used. These yeasts are characteristic of aqueous solutions rich in sugar and in acids and tend to multiply causing evolvement of CO₂ from the metabolism of oxidation of sugars, which phenomenon makes the product unsuitable for consumption.

In a first phase, it is proceeded with sowing of pasteurized orange juice (i.e., initially free of yeasts) while adding to this juice a concentrated inoculum, in order to have, for the examples below, a juice enriched with Saccharomyces cerevisiae yeasts in an amount of 10⁷ microorganisms per milliliter (counted via a standard method for growth on a specific medium of the gelose type).

### COMPARATIVE EXAMPLE

A same volume of orange juice (20 liters) is treated a first time with a treatment device comprising a treatment chamber of volume 5.5 cm³ comprising planar parallel stainless steel electrodes at a flow rate of 25 L/h.

The electric signal delivered between the electrodes is a signal with exponential decrease characteristic of the discharge of a capacitor into the electric resistance formed by the liquid vein present in the chamber. The peak value of the electric field is initially adjusted to a value close to 47 kV/cm producing an energy per pulse close to 5.5 J/pulse. The time constant τ of the pulses defined by τ=RC is close to 700 ns, with R being the electric resistance between the electrodes and C the value of the capacitance of the discharge capacitors.

The juice treated a first time (nᵣ=1 treatment) is recovered in a carboy and stored in an intermediate way and then is subsequently treated a second time by having it again pass into the treatment system, under the same operating conditions (nᵣ=2 treatments) as during the first treatment.

Without taking into account the delay required for the intermediate stagnant storage, the total treatment time is 1 h 36 mins.

The total number of pulses (nₜₒₜₐₗ) (each corresponding to the simple discharge of a capacitor) to which is subject each liquid unit element (each element corresponds to the volume of the treatment chamber), during the first treatment is equal to 120 and also to 120 during the second treatment, which brings the final number of pulses to 240. The characteristics of the pulses are transferred into the table below, i.e. a peak field value of 46.6 kV/cm and a specific energy delivered to each liquid unit element, based on 1 cubic meter (1 m³), of 120 MJ/m³, i.e. 240 MJ/m³ for both treatments.

The characteristics of each treatment appear in Table 1 below.

| | | | E | W_{spec} | |
|---|---|---|---|---|---|
| nᵣ | nₜₒₜₐₗ | Q (L/h) | (kV/cm) | (MJ/m³) | Log N/N₀ |
| 1 | 120 | 25 | 46.6 | 120 | −1.87 |
| 2 | 240 | 25 | 46.6 | 240 | −4.4 |

nᵣ corresponding to the treatment number of the liquid to be treated;
nₜₒₜₐₗ corresponding to the total number of pulses to which is subject each liquid unit element (each element corresponds to the volume of the treatment chamber);
Q (in L/h) corresponding to the supply rate of the liquid;
E (in kV/cm) corresponding to the peak value of the electric field released by each of the electric pulses;
W_{spec} (in MJ/m³) corresponding to the specific energy delivered at each liquid unit element, reduced to one m^{3;}
Log N/N₀ corresponding to the decimal logarithm of the ratio of the number of microorganisms after treatment (N) over the number of microorganisms before treatment (N₀).

From this table, it appears that the effect of each treatment is additive in the sense that each treatment reduces the number of microorganisms by the order of 2 decades. By extrapolation, it may be considered that 3 treatments would have allowed the Log of the population of microorganisms to be lowered by 6 at the expense of a specific energy of 360 MJ/m³.

### EXAMPLE OF THE INVENTION

A same batch of 20 L of orange juice as in the comparative Example above is prepared.

This juice feeds the treatment device in an amount of 20 L/h.

The treatment device is similar to the one used for the application of the comparative Example discussed above except that a circulation loop is provided, placed at the outlet of the treatment chamber of volume 5.5 cm³. The characteristics of the circulation loop are the following: a diameter of 8 mm and a volume of 50 mL. The circulation rate in this loop is set to 360 L/h, which means that the introduction flow rate into the chamber is 380 L/h.

Different tests were conducted wherein:

the same volume of juice is treated (20 L);

the juice supply flow rate is set to 20 L/h while the drawing-off flow rate from the circulation loop is set to 360 L/h;

the treatment period is 1 hour;

the peak value of the electric field is initially adjusted to a value close to 48 kV/cm, producing an energy per pulse close to 5.8 J/pulse comparable to the value of the comparative Example above;

the total number of pulses delivered to each liquid unit element (which corresponds to the volume of the treatment chamber) has a different determined value for each of the tests, which corresponds to a given specific energy.

The operating parameters of the tests applied appear in Table 2 below.

| | | W_{spec} | | | Log |
|---|---|---|---|---|---|
| Test | nₜₒₜₐₗ | (in MJ/m³) | N₀ | N | (N/N₀) |
| 1 | 59 | 62 | 2.2*10⁶ | 2.2*10⁵ | −1 |
| 2 | 79 | 80 | 2.2*10⁶ | 7.75*10³ | −2.45 |
| 3 | 99 | 99 | 2.2*10⁶ | 1.5*10² | −4.17 |
| 4 | 119 | 118 | 2.2*10⁶ | 1 | −6.34 |

nₜₒₜₐₗ corresponding to the total number of pulses delivered to each liquid unit element (which corresponds to the volume of the treatment chamber);
W_{spec} (in MJ/m³) corresponding to the specific energy delivered to each liquid unit element reduced to one m^{3;}
N₀ corresponding to the number of microorganisms before treatment;
N corresponding to the number of microorganisms after treatment.

From this table it emerges that an inactivation greater than 2.4 Log is obtained for a specific energy of 80 MJ/m³ (while in the case of the comparative Example, a specific energy of more than 120 MJ/m³ was necessary for obtaining such inactivation), that an inactivation greater than 4 Log is obtained for a specific energy of 99 MJ/m³ and that for a specific energy of 118 MJ/m³, an inactivation of 6.34 Log is obtained (while for a specific energy of 120 MJ/m³, only an inactivation of the order of 2 Log was obtained).

FIG. 3 represents a graph illustrating Log (N/N₀) versus the specific energy W (in MJ/m³) for the values obtained for the comparative Example (curve a) and the Example of the invention (curve b).

As a conclusion, it emerges that inactivation by 4 Log (i.e. 10,000 times less living microorganisms after treatment than before) may be obtained with 2.4 times less energy within the scope of the Example of the invention. It also emerges that for a same value of specific energy (in this case 120 MJ/m³) a gain of 4 Log is obtained within the scope of the Example of the invention with respect to the comparative Example.

By establishing a circulation loop, it is thereby possible to access results which are superior to those obtained by multiple passages of the same volume of juice to be treated as this is explained in the comparative Example.

Thus, the method of the invention gives the possibility of contemplating a significant reduction both in investment costs and in operating costs while reducing the risk of reviving microorganisms.

As compared with a method not including any circulation loop, the dead space is minimized (i.e. a stagnant volume of treated liquid, as this is the case of the comparative Example, where the treated liquid before its second passage is stored in an intermediate tank).

The originality of the invention also comes from the unexpected effect of the method, wherein the circulation loop not only acts as a mixing apparatus or for increasing the hydraulic conditions.

In the following, the difference in cell mortality is compared for comparable values of specific energy.

The treated product consists of orange juice contaminated by yeast similar to the preceding ones. The product is treated in the same treatment chamber for a peak value of the electric field of 48 kV/cm, producing an energy per pulse equal to 5.7 J. The inactivation results are prepared for a specific provided energy of 100 MJ/m³ (i.e. a total number of pulses of 100).

In a first case, in the absence of a circulation loop with a load of 20 L and a supply flow rate of 25 L/h, by extrapolating the values of the curve illustrated by FIG. 3, the inactivation is 1.5 Log.

In a second case, still in the absence of a circulation loop, with a load of 500 L with a supply flow rate of 500 L/h, an experimental value Log N/N₀=2 is obtained from better hydrodynamic conditions equivalent to better stirring.

If reference is made to FIG. 3, with a load of 20 L and a supply flow rate of 20 L/h, when the circulation loop is applied, for a total flow rate crossing the treatment chamber of 380 L/h (360 L/h in the loop and 20 L/h from the supply), i.e. hydraulic conditions slightly less favorable than at 500 L/h, an inactivation Log N/N₀ of 5 is obtained. Therefore, the favorable effect on cell mortality therefore essentially stems from the number of passages through the circulation loop and not from the hydraulic conditions.

The results appear in Table 3 hereafter.

| | | Introduction | | |
|---|---|---|---|---|
| | | flow rate into | | |
| Supply flow | Circulation | the chamber | W_{spec} | Log |
| rate Q (L/h) | loop | (in L/h) | (in MJ/m³) | (N/N₀) |
| 25 | No | 25 | 100 | 1.5 |
| 500 | No | 500 | 100 | 2 |
| 20 | Yes | 380 | 100 | 5 |

Several circulation loops may be provided, for example 2 or 3 circulation loops.

In Table 2 above, it may be observed that for a specific energy of 118 MJ/m³, with a single circulation loop, the microorganisms quasi move through without any risk of passing with a hydraulic short circuit, i.e. in this case, with a passage probability close to 1 over 2*10⁶. If a second circulation loop is added, everything being moreover equal, the direct passage probability of a microorganism will be close to 1 over (2*10⁶)², i.e. 1 over 4*10¹². If a third circulation loop is added, the direct passage probability of a microorganism will be close to 1 over (2*10⁶)³, i.e. 1 over 8*10¹⁸.

Thus, with a method according to the invention, from a supply liquid, conventionally contaminated with an amount from 10⁶ to 10⁷ microorganisms per milliliter, it is thereby almost possible to cancel any probability of passage of a microorganism into a hydraulic short circuit and to therefore be protected from any risks of reviving a pasteurized liquid by pulsed electric fields.

1. A method for membrane permeabilization of biological cells contained in a product, the method being applied in a treatment device comprising at least one treatment chamber emitting a pulsed electric field, the method comprising:
supplying the treatment device with a product comprising the biological cells at a predetermined supply flow rate from a supply unit comprising the product;
introducing the product comprising the biological cells to the treatment chamber at an introduction flow rate;
treating the product introduced into the chamber with a pulsed electric field; and
feeding back at least a portion of the treated product from the outlet of the chamber at a feedback flow rate to a point that is upstream from the chamber and downstream from the supply unit, wherein the introduction flow rate includes the supply flow rate and the feedback flow rate.

2. The method according to claim 1, wherein the biological cells are selected from the group consisting of prokaryotic cells, and eukaryotic cells, and wherein the biological cells are one of live cells, dead cells, whole cells, partial cells, cells of animal origin, cells of vegetable origin, or combinations thereof.

3. The method according to claim 1, wherein the biological cells stem from unicellular or pluricellular organisms selected from the group consisting of bacteria, fungi, yeasts, molds, and algae.

4. The method according to claim 1, wherein the pulsed electric field is materialized as electric pulses resulting from electric discharges with a duration having a range from about 50 nanoseconds to about 1 millisecond, the electric pulses delivering a voltage producing a peak value of the pulsed electric field ranging from about 5 kV/cm to about 200 kV/cm.

5. The method according to claim 1, wherein the product is subject to turbulent hydraulic conditions.

6. The method according to claim 1, wherein the feedback is carried out by means of at least one circulation loop connecting a downstream portion of the treatment chamber to an upstream portion of the treatment chamber.

7. The method according to claim 1, wherein the feedback flow rate is greater than the supply flow rate.

8. The method according to claim 7, wherein the feedback flow rate is from about 2 to about 100 times greater than the supply flow rate.

9. The method according to claim 7, wherein the feedback flow rate is about 2 to about 20 times greater than the supply flow rate.

10. The method according to claim 1, wherein the treated product is not subjected to any stagnant storage prior to the feedback.

11. The method according to any of the preceding claim 1, further comprising extracting the treated product from the treatment device at an extraction flow rate corresponding to the supply flow rate.

12. The method according to claim 1, wherein the product to be treated is a liquid comprising biological cells, a mud containing biological cells or a pluricellular organism.

13. The method according to claim 1, wherein the product to be treated is a liquid comprising organites or molecules from biological cells.

14. The method according to claim 12, wherein the liquid is selected from the group consisting of water, liquid effluents, liquid muds from sewage works, fruit juices, milk, liquid eggs, sauces, soups, stewed fruit, and purees.

15. A method for pasteurizing or sterilizing a product comprising biological cells to be removed comprising including the method as defined according to claim 1.

16. The method according to claim 15, wherein the product is a fruit juice comprising yeasts as biological cells.

17. The method according to claim 16, wherein the yeast comprises Saccharomyces cerevisiae.

18. The method according to claim 1, wherein the product is a liquid.

19. The method according to claim 12, wherein the pluricellular organism comprises a fruit.

20. The method according to claim 13, wherein the biological cells include at least one of mitochondria, DNA, and RNA.
